(19) United States
(12) Patent Application Publication
(10) Pub. No.: US 2007/0160978 A1
(43) Pub. Date: Jul. 12, 2007

(54) Method for seriological diagnosis and determination of immunisation status, comprising various controls

(76) Inventor: Claude Escarguel, Sanary Sur Mer (FR)

Correspondence Address: DENNISON, SCHULTZ & MACDONALD, 1727 KING STREET, SUITE 105, ALEXANDRIA, VA, 22314, US

(21) Appl. No.: 10/583,771

(22) PCT Filed: Dec. 23, 2004
(86) PCT No.: PCT/FR04/03364

§ 371 Date: Jun. 21, 2006

(30) Foreign Application Priority Data
Dec. 24, 2003 (FR) 0315327
Aug. 3, 2004 (FR) 0408600

(51) Int. Cl. C12Q 1/70; G01N 33/569; G01N 33/554

(52) U.S. Cl. 435/5; 435/7.31; 435/7.32

(57) ABSTRACT

The invention relates to a method for serological diagnosis by means of indirect immunofluorescence, including the systematic control of the presence of rhumatoid factors and anti-nuclear antibodies in the serum of a patient, and the control of the presence and reactivity of the secondary detection antibodies anti IgM and anti IgG used. According to the invention, a plurality of corpuscular microbial antigens and control antigens comprising non-specific immunoglobulins IgG and IgM and nucleated cells are deposited by a robot and fixed on the solid support by means of physical adsorption. The invention also relates to a method for determining the immunisation status of an individual by dosing the specific IgG type antibodies of vaccine-associated antigens, for a plurality of vaccine-associated antigens, according to the inventive diagnostic method.

The present invention concerns a method and diagnosis kit for human and animal infectious diseases. More particularly, it relates to a diagnosis method for human and animal infectious diseases which is based on the search for antibodies in the patient's serum that are specific to the infectious microbial agent, namely a bacterium, virus, parasite or fungus responsible for a pathology (hereunder designated “microbe”).

The present invention also concerns a method and diagnosis kit for the serological determination of a person's vaccine status.

In the present invention, the term “patient” is taken in its broadest meaning, including human and animal patients. By “animal” is meant more particularly:

birds, further particularly fowl raised for their eggs or meat, and

mammals, in particular dogs, cats, horses, bovines such as calves, cows, ovines such as sheep and caprines such as goats.

This diagnosis method is essential for the diagnosis of microbial infections with difficult sampling, culture and/or identification, in particular obligate intracellular bacteria and viruses and facultative intracellular bacteria of genus Ricksettsia, Coxiella, Bartonella, Tropheryma, Ehrlichia, Chlamydia, Mycoplasma, Treponema, Borrelia and Leptospira for example for which, as microbial antigens, the antigens used consist of whole microbes or fractions or fragments of microbes containing one or more antigens. It is possible mechanically to fragment the microbe by mechanical agitation or sonication for example, or to fragment a microbe by enzymatic process to obtain a fraction conserving the antigens which withstand the serological reaction that is the subject of the present invention. The fractions obtained are separated or purified from the other constituents of the microbe and its culture medium using a suitable method e.g. centrifuging or filtration. In this case the term “antigen fraction” or “purified antigen” is used. The whole microbe or any fraction of the microbe is called hereunder a “particulate or corpuscular antigen” in that the whole microbe or one of its fractions cannot be placed in solution by dissolution but solely in suspension in a suitable fluid. The microbes or their fraction remain visible as particles that can be individualized under microscopic observation by optic microscope or electronic microscope for example.

Technically, microbial serology consists of detecting in the patient's serum an antigen-antibody reaction in which the antigen is represented by all or a fraction of the infectious microbial agent to be detected, and the antibody is represented by human or animal immunoglobulins specific to said infectious microbial agent present in the patient's serum. It may be quantified by successively testing a series of increasing 2-fold or 10-fold dilutions of the patient's serum from a first dilution of 1:16 or 1:50 of the patient's serum.

According to the inventive method, the antigen is deposited on a solid support of glass slide type or microtitre plate to implement detection techniques via immunodetection, in particular immunofluorescence, or by enzymatic technique in particular of ELISA type. These detection techniques are well known to those skilled in the art and comprise the successive steps of:

1. decomplementing the serum by heating to 56° C. for 30 minutes,

2. contacting the antigen corresponding to the infectious microbial agent attached to the solid support with the patient's serum then incubating under conditions of time, temperature, hygrometry, mechanical agitation and ionic strength of the medium allowing the antigen-antibody reaction,

3. careful, thorough washing to remove excess patient serum that is not attached to the solid support,

4. applying a secondary detection antibody which is an animal immunoglobulin directed against the immunoglobulins of the patient species under consideration, e.g. for a human patient a goat anti-human immunoglobulin conjugated with a fluorochrome substance, generally fluorescein iso-thio-cyanate or an enzyme, generally a peroxidase, and incubation under conditions of time, temperature, hygrometry, mechanical agitation and ionic strength of the medium enabling the antigen-antibody reaction,

5. careful, thorough washing to remove the excess labelled immunoglobulin that is not attached

6. detection of a reaction by reading with appropriate apparatus in relation to the marker used, such as a fluorescence microscope or microarray for the indirect immuno fluorescence technique, or an optical density reader for the enzymatic detection technique of ELISA type. Reading of the reaction may be by naked eye, or after digital acquisition of the gel and analysis using densitometry software or any suitable software for image processing.

The present invention more particularly concerns detection and assay methods in which the presence is detected, and preferably the quantity is assayed, of immunoglobulins of class M (IgM) and G(IgG) specific to a microbial antigen characteristic of a microbe. These determinations give more precise and complete data required for establishing aetiology and following the development of some infectious diseases. In general IgMs are seen at an earlier stage. As for IgGs, these can be used to determine the serological status of the patient against a given microbe, with a view to making a serological diagnosis of the infection or determining the extent of body protection against this microbe.

The specificity of the infectious antigen/serum antibody reaction determines the specificity and positive predictive value of the serological test based on this reaction, and any fixing of a non-specific antibody onto the microbial antigen limits the specificity and predictive value of the serological test. The presence in the serum to be tested of rheumatoid factors or anti-nuclear antibodies is a source of non-specific fixing of antibodies on the microbial antigen as is explained below.

These rheumatoid factors are class M immunoglobulins recognizing the Fc fragment of IgGs of different species including human IgGs (IgM anti-IgG).

The presence of rheumatoid factors in a patient's serum is responsible for false positive results when detecting IgMs specific to a microbial antigen during serological tests for the indirect diagnosis of infectious diseases. These rheumatoid factors fix to IgGs specific to the microbial antigen contained the patient's serum and therefore appear as false positives when detecting specific IgMs in a serological reaction using the microbial antigen in detection tests by agglutination.

It is possible to eliminate rheumatoid factors from the serum by adsorption using non-specific human IgGs that are added to the sample in excess. These added IgGs are modified by heating or treatment with glutaraldehyde so that they do not interfere with the detection of the specific IgGs. This addition of IgGs causes precipitation after complexing with the anti-IgG substances contained in the sample. But in this case, the specific IgGs and IgMs contained in the sample may also be denatured by the modification treatment undergone by the IgGs added to the sample.

Also, to date there exists no systematic control of the efficacy of this adsorption before conducting the serological reaction in IgM and IgG detection and assay methods.

Also, the presence in the patient's serum of anti-nuclear antibodies limits the specificity of the microbial antigen/serum antibody reaction. Anti-nuclear antibodies are antibodies of IgG type directed in non-specific manner against the group formed of the DNA and nuclear proteins of the chromosome of eukaryote cells and microbes, called histones. These anti-nuclear antibodies therefore fix non-specifically to any eukaryote cell, including fungi and parasites, and on any microbe, bacterium, DNA virus, parasite or fungus. This phenomenon leads to a non-specific positive reaction during serological tests entailing the detection of IgG specific to a bacterium, DNA virus, fungus or whole parasite or which contain DNA/histone complexes as microbial antigen, by means of anti-IgG detection antibodies in a serum containing anti-nuclear antibodies.

The detection of anti-nuclear antibodies in the serum to be tested, conducted using an immunodetection technique by immunofluorescence has been described [Fritzler M J, In: Manual of Clinical Laboratory Immunology, Fourth edition, Rose N R, Conway de Macario E, Fahey J L, Friedman H, Penn G M, (eds) American Society for Microbiology, Washington, D.C. 1992, pp. 724-729] which uses mammalian cells including human cells such as Hep-2 cells as antigen. In this method, the cells are formed of a mat of confluent cells deposited and “read” manually. This depositing of confluent cells is too viscous for robot depositing using a syringe type robot arrayer, and reading cannot be automated since only manual depositing enables the depositing of a large quantity of cells so as to guarantee sufficient detection of the cell/anti-nuclear antibody reaction. In these publications, no mention is made of the need to conduct systematic control of the presence of anti-nuclear antibodies in immunodetection tests of microbial antigens. In addition, detection with confluent cells deposited manually on a solid support is not applicable for routine laboratory tests.

There is not therefore any method published to date for the control or elimination of anti-nuclear antibodies before conducting a serological test to diagnose infectious diseases so as to guarantee the absence of any false positive reaction in the results, and which can be given routine laboratory application. This is why no serology protocol published in the reference manuals and no marketed serological test systematically includes the screening of anti-nuclear antibodies or of rheumatoid factors as prerequisite for performing or interpreting serology.

In practice, at the present time, in immunodetection tests routinely used in laboratories it is only possible at the most to detect false positives due to the presence of rheumatoid factors (or anti-nuclear antibodies) by conducting tests on a plurality of microbial antigens whose concomitant presence is not probable. But it will be easily understood that this type of verification considerably increases costs in terms of equipment, labour and time consumption.

Methods and diagnosis kits are known involving the use of a solid support on which soluble proteins are attached covalent fashion, but these covalent chemical couplings are complex and costly to perform. The non-covalent fixing of soluble proteins or particulate antigens on a solid support has been proposed by physical or physicochemical adsorption on the support, under test protocols for immunodetection on a solid support, but the stability of fixing is insufficient. One difficulty lies in the fact that it is necessary previously to wash the solid support thoroughly to eliminate residues of labelling elements which may interfere with the reading of results, whereas such washing renders the physical adsorption of the substances on the solid support too unstable. Another difficulty lies in the fact that it is not possible to deposit corpuscular antigens using robot arrayers, whether or not they are whole or partial cells or bacteria as mentioned above.

A first purpose of the present invention is to provide a reliable immunodetection test for microbial antigens, using a method and tools that are easy to implement and produce for the routine laboratory application of tests conducted in series for cases when the test serum could contain rheumatoid factors or anti-nuclear antibodies, and a method with which it is possible to overcome the need for positive and/or negative control serum samples.

A further purpose of the present invention is to provide a method for preparing a solid support allowing robot arrayer depositing and non-covalent attachment of control antigens by physical adsorption on the support to control the presence of rheumatoid factors, antinuclear antibodies and microbial antigens, consisting of proteins or particulate antigens as explained above, and automated reading to verify the absence of rheumatoid factors and anti-nuclear antibodies and to read results of the immunological reaction in a patient serum test involving a microbial serological reaction of specific IgMs or IgGs by adsorption with the particulate or corpuscular microbial antigen deposited on the solid support.

Similarly, no marketed serological test systematically includes control of the added presence and reactive value (or immunological reactivity) of the anti IgM or IgG secondary detection antibodies used, and a further original purpose of the present invention is to provide a test comprising this reactivity control.

For this purpose the present invention provides an in vitro serological method for diagnosing a microbial agent by immunodetection, in which the presence is detected, and preferably the quantity is assayed, of patient immunoglobulins of both classes M and G or only of class G specific to a microbial antigen characteristic of said microbial agent, in a serum sample of the patient to be tested, by detection and preferably quantification of an immunological reaction complex between said microbial antigen to be detected and a said specific, class M immunoglobulin for the assay of IgM and/or respectively a said immunoglobulin of class G for the assay of IgG, using a first detection substance and/or respectively second detection substance, preferably an antibody only reacting with a said immunoglobulin of the patient species of class M and/or respectively class G, characterized in that:

1/The steps are performed in which:
said serum sample to be tested is contacted with said first and second detection substances, or only said second detection substance, and with at least one solid support on which the following antigens have been attached:
   a first control antigen corresponding to a non-specific class G immunoglobulin of the patient species, and
   a second control antigen containing DNA/histone complexes, preferably all or part of nucleate cells comprising nuclei of nucleate cells, more preferably continuous line cells, further preferably non-confluent cells in suspension, and
   optionally, a third control antigen corresponding to a non-specific class M immunoglobulin of the patient species, the presence of said third control antigen being necessary in the event of IgM assay, and
   at least one said microbial antigen, and
a series of controls is conducted comprising:
   a—controlling the reactivity of said second detection substance by verifying whether said first control antigen reacts with said second detection substance, and optionally control of the presence of rheumatoid factors in said serum sample by verifying whether the first control antigen reacts with said serum sample and said first detection substance, in the event of IgM assay,
   b—controlling the presence of anti-nuclear antibodies in said serum sample to be tested by verifying whether said second control antigen reacts with said serum sample and second detection substance,
   c—controlling the reactivity of said first detection substance by verifying whether said third control antigen reacts with said first detection substance, in the event of IgM assay, and
   d—controlling the presence of a human serum in the sample to be tested, and

2/A reaction result between said microbial antigen, said serum sample and a said detection substance is only taken into account if the control of the presence of a human serum is positive and if the following accumulative conditions are met determining the absence of anti-nuclear antibodies and the reactivity of said second and, when applicable, said first detection substance, and optionally absence of rheumatoid factor:
a—said first control antigen reacts with said second detection substance,
b—said second control antigen does not react, and
c—when applicable, if said third control antigen reacts with said first detection substance, in the event of IgM assay.

If the sample contains rheumatoid factors (IgM anti-IgG) these may react with the immunoglobulins (IgG₁) attached to the solid support, to form the following complex with said first detection substance (Ac₁ anti IgM*¹): (S-IgG₁-IgM anti IgG-Ac₁ anti IgM*¹) (S=solid support).

When the absence of any rheumatoid factor is established, the detection of a reaction between said first detection substance and said microbial antigen (Agmic) is effectively proof of the presence in the tested serum of class M immunoglobulins specific to the microbial antigen (IgM anti Agmic) through formation of the complex (S-Agmic-IgM anti AGmic-Ac₁ anti IgM*¹) and not of the presence of IgG specific to said microbial antigen (IgG anto Agmic) which could, in the presence of a rheumatoid factor, form a false positive complex (S-Agmic-IgG anti Agmic-IgM anti IgG-Ac₁, anti IgM*¹).

In addition, as explained below, when said first antigen consists of an IgG of the patient species, human in particular, this enables verification of the presence and reactivity of a said second detection substance specifically recognizing the IgGs of the serum of the patient species.

For a human patient, as said second control antigen advantageous use be made of non-confluent human fibroblast cells in suspension, in particular HL60 cells.

If the sample to be tested contains anti-nuclear antibodies (which are IgGs, human in particular) these may react with said second antigen (Ag₂) and be detected by said second detection substance (Ac₂ anti IgG*²) since the latter is a substance reacting with IgGs of the patient species, human in particular, and form the complex (S—Ag₂-IgG anti Nucl-Ac₂ anti IgG*²).

The detection of a reaction between said first detection substance and said second antigen (Ag₂) attached to the solid support, necessarily means that a complex has formed with anti-nuclear antibodies (Ac anti nucl.) as per: (S—Ag₂-IgG anti nucl.-IgM anti IgC—Ac₁ anti IgM*¹) and hence that the sample contains both the rheumatoid factor and anti-nuclear antibodies.

Once the absence of anti-nuclear antibodies has been established, the detection of a reaction of said second labelling substance with said microbial antigen is indeed evidence of the presence of IgG specific to said microbial antigen and of the formation of a complex (S-Agmic-IgG antiAgmic-Ac₂ anti IgG*²) and not of a false positive complex resulting from the reaction of anti-nuclear antibodies with the microbial antigen as per the complex (S-Agmic-Ac anti nucl.-Ac₂ anti IgG*²).

The reactivity of said second detection substance added to said serum sample to be tested is also verified, in the absence of rheumatoid factor and anti-nuclear antibody in said sample. If said second detection substance is truly reactive in the absence of rheumatoid factor, the following complex (S-IgG₁-Ac₂ anti IgG*²) should be detected on said first antigen (IgG₁). In this case, the absence of reaction by said second detection substance with said second antigen provides good evidence of the absence of anti-nuclear antibodies.

If said first detection substance is present and is reactive, a complex S-IgM₁-Ac₁ anti IgM*¹ should be detected on said third control antigen (IgM₁). Thereafter the non-detection of a complex containing said first marker at said first antigen (IgG₁) and when applicable at said second antigen attached to the solid support, is indeed evidence of the absence of rheumatoid factor.

More particularly, a robot is used to deposit a solution of IgG and IgM of the patient species, human in particular, γ-specific (specific to the gamma chain of the immunoglobulins of the patient species, human in particular) as said first and third control antigens.

The present invention therefore allows the systematic detection of rheumatoid factors, anti-nuclear antibodies, and the systematic control of the reactivity of the detection anti-immunoglobulin antibodies in a serum used for serological diagnosis by indirect immunofluorescence, after robot depositing on a solid support of class G and M immunoglobulins of the patient species, human in particular, and of nucleate cells of the patient species.

Another frequent error when performing serological tests, in particular battery serological test made on a large number of sera to be tested, is due to the faulty adding of the sera to be tested, in particular when pipetting. These errors occur in particular at the steps which involve displacement of the sample to be tested, in particular by pipetting, since it is possible that some containers in particular those containing the solid support on which the antigen to be detected is deposited, may inadvertently not be filled with the serum of the patient species, human in particular, to be tested. It is known that serum pipetting carries an error risk of 1% related to a purely technical problem through non-pipetting by the pipette, or a human error through inadvertent omission when pipetting.

These errors require the introduction of controls when performing the reaction. The systematic incorporation during each new handling operation of a negative control serum i.e. not containing antibodies specific to the antigen to be tested, enables the interpretation of positive reactions. Similarly, the incorporation of a positive control serum i.e. containing the antibody specific to the tested antigen and having a known titre can be used to verify the quality of the antigen and conjugate immunoglobulin.

However no control currently exists to verify whether the serum to be tested has in fact been added to the serological test. However, should by inadvertence the serum to be tested not be added to the serological test, the bacterial antigen/serum antibody reaction will certainly not take place and the test will be falsely interpreted as negative (false negative). In the present invention, advantage is drawn from the fact that protein A of Staphylococcus aureus has an affinity for sera of animal origin, in particular equine, bovine, pig, rabbit, guinea-pig, mouse; to a lesser extent hamster, rat and sheep. On the other hand, chick and goat sera do not react with protein A. Protein A is a polypeptide of 42 kDa and is a constituent of the wall of Staphylococcus aureus strain; similar but different proteins are characterized on the surface of bacteria of genus Streptococcus [Langone J J, Adv. Immunnol. 1982,32:157-251]. This property of protein A of Staphylococcus aureus has already been used in a serological test in man for the serological diagnosis of infectious endocarditis [Rolain J M, Lecam C, Raoult D. Simplified serological diagnosis of endocarditis due to Coxiella burnetii and Bartonella; Clin. Diag. Lab. Immunol. 2003; 10:1147-8].

The present invention therefore comprises the introduction of a control verifying the addition of the serum to be tested during the bacterial serological reactions.

Insofar as protein A reacts with animal and human immunoglobulins in non-specific manner, even in major infectious pathologies, it is possible to use this protein A as positive control for verifying the addition of a serum of the patient species, human in particular, to the sample to be tested.

More precisely, according to the invention, it is controlled that said tested sample indeed contains a serum of the patient species, by detecting whether immunoglobulins of the patient species react with a fourth control antigen containing protein A of a Staphylococcus aureus bacterium, preferably said fourth antigen being a whole Staphylococcus bacterium, by contacting said sample with a solid support on which a said fourth control antigen is attached, in the presence of said second detection substance which is a substance reacting with an immunoglobulin of the patient species and not reacting with said fourth control antigen, preferably an anti-immunoglobulin antibody of the patient species not reacting with said fourth control antigen, the control of the presence of a serum being positive if said fourth antigen reacts with said serum sample and said second detection substance.

In one advantageous embodiment, said fourth antigen is a whole Staphylococcus aureus bacterium containing protein A. More particularly use may be made of the Staphylococcus aureus bacteria deposited in public collections such as the bacteria deposited with A.T.C.C. under n° 29213 and the C.N.C.M collection at Institut Pasteur (France) under number 65.8T, as described in the above-mentioned publication [Rolain J M, Lecam C, Raoult D. Simplified serological diagnosis of endocarditis due to Coxiella burnetii and Bartonella. Clin. Diag. Lab. Immunol. 2003 ; 10:1147-8].

Also, in addition to type strains, any bacterial strain identified as Staphylococcus aureus may be used as said fourth control antigen.

Preferably, said second detection substance is an animal immunoglobulin, preferably a goat or chicken immunoglobulin.

Advantageously a single solid support is used contacted optionally simultaneously with said first and second detection substances containing a first and respectively a second labelling element, the second labelling element emitting a signal different to the first labelling element, preferably said first and second detection substances containing a first and respectively a second antibody only reacting with a said immunoglobulin of the patient, respectively of class M and of class G.

Further advantageously, optionally, the two said first and second detection substances are goat or chicken immunoglobulins, respectively anti-IgM and anti-IgG.

Further advantageously, the said detection substance(s) are an antibody (antibodies) conjugated with a labelling element containing a fluorescent substance (substances). In particular it is verified whether a reaction product complex can be detected by fluorescence between said fourth antigen and said third detection substance.

Advantageously in an inventive diagnosis method, the said immunoglobulin of the patient species specific to said microbial antigen is detected and optionally assayed in the sample to be tested, and the immunological reactions between said control antigens and said detection substances are read off by automated reading using reading equipment appropriate for said labelling elements, preferably equipment which reads a fluorescent signal of a fluorescent substance corresponding to the labelling elements of said detection substances.

As labelling element of said detection substances, advantageously enzymatic, radioactive or fluorescent labelling is used, fluorescent labelling being preferred.

The expression “fluorescent labelling” means that the antibody has been made fluorescent by coupling or complexing with a suitable fluorescent agent such as fluorescein iso-thio-cyanate or any other substance emitting radiation detectable after its illumination, each substance being characterized by the wavelength at which it must be illuminated (excitation wavelength) and the wavelength of the radiation it emits (emission wavelength).

The expression “radioactive labelling” means that the antibody carries a radioactive isotope allowing its assay by counting its associated radioactivity, the isotope possibly being carried either by an element of the antibody structure e.g. constituent tyrosine residues, or by an appropriate radical that has been attached to it.

The expression “enzymatic labelling” means that the specific antibody is coupled or complexed to an enzyme which, associated with the use of suitable reagents, allows quantitative measurement of this specific antibody.

The substrate and reagents are chosen so that the end product of the reaction or sequence of reactions caused by the enzyme with these substances, is:

either a stained or fluorescent substance which diffuses in the liquid medium surrounding the tested sample and which is either finally measured by spectrophotometry or fluorimetry respectively, or is assessed by eye optionally with respect to a range of calibrated hues,

or a stained, insoluble substance which is deposited on the tested sample and can be measured either by reflection photometry or by eye assessment optionally with respect to a range of calibrated hues.

If a detection substance made fluorescent is used, the fluorescence associated with the tested sample is read directly on suitable equipment able to detect radiation at the emission wavelength and to quantify the same.

According to one advantageous embodiment, said control antigens and microbial antigens are attached to the solid support by physical adsorption. And further advantageously, said microbial antigen is a particulate or corpuscular antigen such as described previously consisting of a whole inactivated microbe or microbe fraction.

As explained below, the inventors have developed conditions for depositing said control and microbial antigens on a solid support, which can be reconciled with depositing by mere physical adsorption, and more particularly when said microbial antigens are corpuscular antigens.

In one particular embodiment, said microbial antigen is chosen from among micro-organisms comprising a bacterium, a virus, a parasite or a fungus.

More particularly, said microbial antigen is an intracellular bacterium and in particular said microbial antigen is chosen from among the bacteria of genus Rickettsia, Coxiella, Bartonella, Tropheryma, Ehrlichia, Chlamydia, Mycoplasma, Treponema, Borrelia and Leptospira, this list not being exhaustive.

Further particularly, said microbial antigen is a bacterium responsible for endocarditis.

In another embodiment, said microbial antigen is viral antigen, in particular a virus chosen from among the H.I.V., C.M.V. or Epstein-Barr viruses, this list not being exhaustive.

Preferably, a method of the invention comprises the characteristics according to which:

the detection and preferably the assay is performed of both class M and G patient immunoglobulins specific to a microbial antigen,

at least one said microbial antigen and said first, second and third and fourth control antigens are attached to one same solid support, and

for the detection of the different said microbial antigens, the same said first and second detection substances are used with different labelling elements, said first and second detection substances being animal immunoglobulins not reacting with said fourth antigen, preferably goat or chicken immunoglobulins in human patients.

In this last embodiment, a protocol for a succession of controls is as follows:

1) It is verified that said fourth control antigen containing protein A reacts with said second detection substance. If not, the test is stopped i.e. this sample is not taken into account.

2) If said first control antigen (IgG₁) reacts with said first detection substance (Ac1 anti IgM*1) the serum sample contains rheumatoid factors, therefore once again the detection and quantification tests of the specific IgMs are not taken into account.

3) If said first control antigen does not react with the second detection substance (Ac₂ anti IgG*²), said second detection substance is not present or is not reactive. The test result concerning the detection of IgGs specific to the microbial antigen is not taken into account.

4) If said first control antigen reacts with the second detection substance, there is no rheumatoid factor and said second substance is reactive: the test can be continued i.e. results can be taken into account subject to the following verifications concerning reactions with the second, third and fourth control antigens.

5) If said second control antigen containing a DNA/histone complex reacts with said second detection substance, anti-nuclear antibodies are present and the detection and quantification tests of specific IgGs are not taken into account.

6) If said second control antigen reacts with said first detection substance, rheumatoid factors and anti-nuclear antibodies are present and the test is stopped.

7) If said second control antigen does not react, and if said first and second detection substances are present and are reactive, there are no anti-nuclear antibodies and the test can be continued subject to the following verification.

8) It is verified that said third control antigen reacts with said first detection substance. If said third control antigen (IgM) does not react, said first detection substance is not present or does not react, and the test is stopped.

To conclude, the result of the reaction with said microbial antigen is only taken into account if the following accumulative conditions are met:
said fourth antigen reacts,
said first antigen reacts with said second detection substance,
said second antigen does not react, and
said third antigen reacts with said first detection substance.

In one particular embodiment, said microbial antigen is a vaccine antigen and said immunoglobulin specific to said vaccine agent to be detected is a class G immunoglobulin.

The method then enables determination of the vaccine status of an individual, which is particularly useful to assess the need for a booster vaccine.

Determining the presence and even the level of specific protective antibodies in the serum of a person to be vaccinated is a key element in deciding the administering of a vaccine or booster vaccine. If the person's serum contains antibodies specific to the pathogen at a protective concentration, there will be no benefit but on the contrary a risk in administering a booster vaccine to this person.

In currently proposed methods, the detection of the presence or the measurement of the concentration of antibodies specifically directed against different vaccine antigens is conducted separately for each one. Therefore several serum samples must be taken which are analyzed separately, possibly in several laboratories each having analytical capability for only one vaccine antigen or a limited number of vaccine antigens.

At the present time no kit or automated, reproducible test exists which can be used to determine the vaccine status of a person by determining the titre of specific antibodies against the chief vaccine pathogens. Therefore at the present time no method and kit is available with which it is possible, within a time lapse of less than 24 hours and on a single serum sample, to determine the vaccine status of a person i.e. to detect or determine concentrations of specific IgG antibodies in relation to all currently available vaccines.

A further purpose of the invention is to provide a technique for determining the vaccine status of a person which is simple, quick and low cost and can be used in particular by any laboratory for the simultaneous analysis, from one same serum sample to be tested, of the antibody concentration against a plurality of vaccines currently available, and using a relatively reduced sample volume and hence compatible with samples taken from children including infants. Further particularly, it must be possible to automate the diagnosis technique and to reproduce the technique reliably, both regarding its implementation and the preparation of the solid supports used.

For this purpose, the present invention provides a method in which the vaccine status of a person is determined by detecting, preferably quantifying, IgG serum antibodies specific to vaccine antigens of a plurality of pathogenic agents of bacterial, viral, fungal or parasitical type, by performing the detection, and preferably quantification, of a complex of immunological reactions between each said vaccine antigen and respectively each said antibody specific to said vaccine antigen, which may be present in a human serum sample to be tested, comprising:

1. Contacting one single, same said serum sample to be tested with:

one same solid support on which a plurality of said vaccine antigens is attached corresponding to a plurality of pathogenic agents, and said first, second and preferably fourth control antigens,

in the presence of at least one said second detection substance reacting with at least one said specific antibody and not reacting with any of said vaccine antigens, and

2. Performing at least one said control of the reactivity of said second detection substance using a said first control antigen, and one said control of the presence of anti-nuclear antibodies using at least one said second control antigen, and controlling the presence of human serum in said sample to be tested preferably using a said fourth control antigen.

Also, in known manner, quantification is performed by comparing the signal emitted by the reaction complex: antigen/antibody/detection substance of the tested serum with a reference curve obtained by calibrating control sera containing a known concentration of antibodies to be detected.

By “vaccine antigen” is meant an antigen able to stimulate an immune response by the patient inducing the production of protective serum antibodies, i.e. an antibody binding to the pathogenic agent so that the body comes to be protected against the pathogenic effects of said agent.

Preferably, in the inventive method, one same said first detection substance is used to detect the different antibodies specific to the different vaccine antigens.

Further preferably, and more particularly, a said first detection substance is used which is an anti-IgG immunoglobulin, preferably a goat or chicken immunoglobulin.

One particular embodiment, said vaccine antigens are antigens of pathogenic agents chosen from among the viruses of mumps, rubella, measles, chicken pox, poliomyelitis, yellow fever, tick-borne encephalitis, hepatitis A, hepatitis B, and bacteria of Bordetella pertussis, tetanus and diphtheria

Advantageously, it is determined whether the concentration of said specific antibodies reaches a threshold on and after which said specific antibody has a protective action protecting against the disease determined by the pathogen.

In some cases, it is possible to determine a concentration of specific antibodies imparting protection in nearly 100% of persons vaccinated, the determination of this concentration (or threshold) being made by sero-epidemiological epidemiological studies on vast populations.

The inventive methods are more particularly advantageous when at least one said microbial or vaccine antigen is a corpuscular microbial antigen formed of a whole inactivated microbe or microbe fraction, in particular a vaccine antigen in the form of a viral suspension of whole, living, deactivated viruses.

The inventors have developed a technology with which this type of particulate or corpuscular antigen can be attached to the solid support by mere depositing and physical adsorption or physicochemical binding with the support using an inventive method for preparing the solid support as is explained below. These particulate or corpuscular antigens have the advantage of being clearly visible after depositing on the solid support and, in particular, at the time of reading to detect any immunological reactions.

Therefore, as microbial or vaccine antigens, advantageously antigens are used consisting of whole inactivated microbes or fractions or fragments of microbes containing one or more antigens. It is possible to fragment the microbe mechanically by mechanical agitation or sonication for example, or the microbe can be fragmented using an enzymatic process to obtain a fraction conserving the antigens which withstand the serological reaction that is the subject of the present invention. The fractions so obtained are separated or purified from the other constituents of the microbe and its culture medium using an appropriate method e.g. centrifuging or filtration. In this case the term used is an antigenic fraction of purified antigen. The whole microbe or any fraction of the microbe is hereinafter called “particulate or corpuscular antigen” in that the whole microbe or one of its fractions cannot be placed in solution by dissolution, but solely in suspension in a suitable fluid.

The microbes or their fraction thus deposited are remarkably and clearly visible as individual particles under microscopic examination with an optical or electronic microscope for example.

Technically, microbial serology consists of detecting an antigen/antibody reaction in the patient's serum, in which the antigen is represented by all or fraction of the infectious microbial agent to be detected, and the antibody is represented by the human or animal immunoglobulins specific to said infection microbial agent and found in the patient's serum. It may be quantified by successively testing 2-fold or 10-fold serial dilutions of the patient's serum using a first dilution of 1:16 or 1:50 of the patient's serum.

In one preferred embodiment, for each detection and optional quantification of said vaccine antigen, the following measurements are made:

1—a first measurement of a first value representing the quantity of a first labelling element or “marker”, preferably the first intensity value of a signal emitted by said first labelling element, further preferably fluorescent, said first labelling element binding itself non specifically to any protein in the depositing area of said vaccine antigen, and

2—a second measurement of a second value representing the quantity of a second labelling element emitting a different signal to said first labelling element, preferably a second value of the intensity of the signal emitted by this second labelling element, also preferably fluorescent at a different excitation wavelength to that of the first fluorescent labelling element, said second labelling element being the labelling element of said second detection substance for said vaccine antigen in the depositing area of said antigen, and

3—the ratio between said first and second values is calculated, and

4—the value of said ratio is compared with the value of a reference ratio obtained with a collection of positive and negative reference sera, thereby making it possible to determine, by comparison, whether or not it is necessary to vaccinate the person against said vaccine antigen in relation to the ratio between said first and second values.

The present invention also provides a diagnosis kit which can be used to implement a method of the invention, characterized in that it comprises:

at least one said solid support, preferably a single solid support, on which at least one said microbial antigen and said control antigen(s) are attached, and

said detection substance(s) and reagents used to develop said labelling element(s).

Preferably a kit of the invention comprises:

one same said solid support on which at least one said corpuscular antigen and said control antigens are attached by physical adsorption, and

at least one of a same said first or second detection substance to detect the different microbial antigens.

Advantageously, as solid support in the methods and diagnosis kits of the invention a glass or plastic slide is used, a titre tube or a well of a microtitre plate in plastic, and more particularly as solid support any device may be used adapted for handling cell and bacterial suspensions in particular tubes, glass or polymer slides, shell vials or rigid microtitre plates in polyethylene, polystyrene, chloride.

To implement a method of the present invention, it is possible to take a serum sample by vein puncture using a needle or by sampling capillary blood, in particular from the ear lobe, finger pad, heel pad, coupled to collection on a filter paper disk or preferably directly in a buffer enabling elution of the serum.

According to other advantageous characteristic of the present invention, after collecting capillary blood in a capillary tube with which a known volume of whole blood can be collected, said sample is collected in a flask containing a known volume of elution buffer. This original collection method offers the advantage of rapidity (collection, elution and dilution being performed in one minute). Since the serum is diluted to a known concentration, in particular 1:20 to 1:100, this provides safety for operator handling against risks of accidental exposure to blood such as the transmission of viruses or other blood pathogens.

Also with the present invention it is possible to determine the vaccine status of a person simultaneously against several vaccine antigens, on a single serum sample taken by capillary puncture, within a time lapse of less than 2 hours.

Advantageously, a determined volume of whole blood is collected using a capillary tube in a flask containing a determined volume of buffer enabling serum elution, the serum then preferably being diluted to a determined concentration, preferably 1:100 to 1:20 and a determined volume of serum thus diluted is deposited on the different deposit areas for said control antigens and vaccine antigens on said solid support.

Irrespective of the blood collection mode, “needle sampling” or “capillary puncture” the serum must initially be separated from the blood cells. With capillary puncture this separation is made by eluting with an elution buffer, and it is the product of this elution which is contacted with the slide.

Advantageously therefore the inventive kit comprises a flask containing a determined volume of elution buffer to collect a determined volume of a serum sample to be tested.

A further subject of the present invention is a method for preparing a solid support on which at least antigen is attached chosen from among a said microbial antigen, preference corpuscular, a said first, a said second, optionally a said third and preferably a said fourth control antigen allowing detection by automated reading using a said first and optionally said second detection substance which can be used in a method or kit of the invention, characterized in that said microbial antigens, preferably corpuscular, and said control antigens are deposited by a robot preferably comprising a syringe, said antigens preferably being associated with a dye, further preferably a fluorescent dye, in suspension form at a concentration enabling their visualization after depositing by means of said dye, and also enabling verification of the attachment of said antigens to said solid support.

Preferably, a said microbial antigen and optionally a said second control antigen, and optionally a said fourth control antigen are deposited by robot in the form of a suspension of non-confluent cell corpuscles, whole viruses or whole bacteria or fractions of cells or bacteria.

According to the present invention the inventors, after numerous unsuccessful attempts, have developed conditions for the robot depositing of microbial antigens or corpuscular vaccine antigens (whole inactivated microbe or fraction of whole inactivated microbe) in suspension in a depositing medium. Solely homogeneous solutions of one or more molecules are deposited on solid supports by robot arrayers. For this purpose, the concentration of these corpuscular antigens is calibrated before depositing by counting the inactivated microbial particles by Fluorescence Activated Cell Sorting (FACS-scan) and they are then deposited by robot on a solid support. The designing of these calibrated, robotized deposits entails the determination, by testing, of the optimal concentration for each of the antigens tested, the infra-optimal concentrations giving undetectable deposits, the supra-optimal concentrations leading to the sedimentation during depositing of high density corpuscular antigens of micrometer size such as whole or fractionated bacteria, and hence a substantial variation in the quantity of antigen deposited. Finally, the deposits of corpuscular antigens containing microbial DNA (bacterium, virus, microscopic parasites or fungi) are calibrated by applying a dye preferably a fluorescent dye in particular a molecule such as AMCA which attaches non specifically to the proteins contained in the antigen, or an insertion molecule which attaches non specifically to the DNA by insertion in the double helix. This latter method is preferably used to label cells used as controls on slides. The excitation and emission wavelengths are advantageously chosen in relation to those used by the fluorochrome labelling the detection immunoglobulins. This fluorescent, non-specific labelling of the antigens can be made before or after robot depositing. The fluorescent marker is advantageously chosen for its stability to daylight.

More particularly, in the inventive method for preparing the solid support, the control antigens in the form of a cell suspension are calibrated at a concentration of 10⁷ to 10⁹ cells/ml, and said control agents or microbial antigens in the form of suspensions of bacteria or fractions of bacteria at a concentration of 10⁷ to 10⁹ particles/ml, and the suspensions of whole viruses at a concentration of 10⁹ to 10¹⁰ particles/ml.

Advantageously, said control antigens and microbial or corpuscular vaccine antigens are deposited in a mixture with a protein binder, to stabilize attachment to said solid support.

More particularly, said protein binder is chosen from among the organic binders comprising egg yolk, gelatine, bovine serum albumin or non-human polyclonal IgG, preferably goat.

These protein binders act as biological glue for said antigen on the solid support.

The different binders were tested on a glass slide and the optimal concentrations were determined. In particular bovine serum albumin can be used at a final concentration (volume/volume) of 1 to 5%, a suspension of egg yolk at a final concentration of 1 to 10%, and said goat IgG at a final concentration of 25 to 75%.

The inventors remarked during the different tests conducted that some antigens bound by egg or bovine albumin were washed off during washing steps but that the human IgG immunoglobulins added as control invariably remained attached to the glass slide. It was therefore inferred by the inventors that class G immunoglobulins attached to the glass slide in a manner such that they could withstand the washing steps, and they put forward the hypothesis that these IgG could also be used to attach some vaccine antigens under suitable conditions. The inventors therefore used IgGs of a species other than man in order not to interfere in the serological tests, and the authors thereby determined the remarkable properties of goat IgGs as biological glue allowing the attachment of particulate antigens, in particular particulate or corpuscular vaccine antigens.

Preferably, and more particularly, said corpuscular vaccine antigen is deposited on said solid support formed of a glass slide, in a mixture with an immunoglobulin of goat polyclonal IgG type.

A further subject of the present invention is a method for preparing a solid support characterized in that prior washing of said solid support is performed with a solution of an ethanol/acetone mixture preferably 50/50, then said antigens are deposited and their attachment stabilized by physical adsorption on said solid support by treatment with alcohol, preferably methanol or ethanol, which alcohol is subsequently removed, and preferably the attachment of said antigens is verified by staining in particular by fluorescent marking that is non specific to the proteins or DNA as explained above.

This prewash solution is used to clean the support of any trace of detection substance or other residual and in particular to remove any fluorescence while maintaining the possibility of physical adsorption of the support for the said subsequently deposited antigens.

Stabilization treatment with alcohol stabilizes attachment by physical adsorption both of the proteins, such as IgGs, and of the particulate antigens.

The determination of a suitable prewash for the solid support, in particular the glass slide, required numerous tests. The objective of this treatment is to clean this support thoroughly so as to remove fluorescent artefacts whilst preserving subsequent attachment of the antigens in a manner compatible with their conservation mode, but also by preserving if not the integrity at least the immunological reactivity of the antigens. It was shown, after numerous unsuccessful attempts, that the rinsing and cleaning of slides in an aqueous phase does not allow subsequent attachment of the antigens to be deposited; the same applies to rinsing with surfactant molecules such as Tween 20. Cleaning with alcohols was insufficient to remove most of the fluorescent artefacts. It is therefore after multiple tests that a solid support cleaning protocol could be optimized, in particular for a glass slide, using a mixture of methanol 50%—acetone 50% followed by drying in air.

However, even in this case, it remains advantageous to complete attachment by physical absorption with a cross-linking treatment, in particular a chemical treatment with a bifunctional covalent coupling agent such as glutaraldehyde or activated diacid derivatives, succinimic acid in particular, known to persons skilled in the art for ensuring covalent bridges between said control and microbial antigens with the solid support.

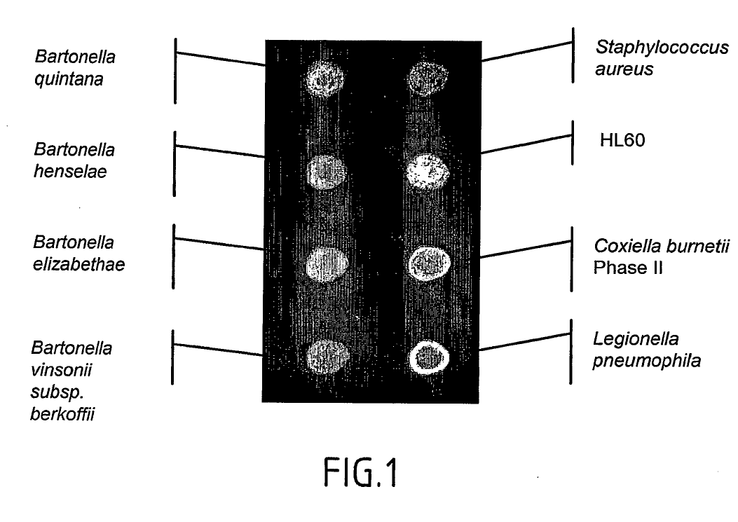

Other characteristics and advantages of the present invention will become apparent in the light of the following examples which refer to FIGS. 1 to 3.

FIG. 1 is a slide stained with a fluorescent dye, enabling visualization of all 8 deposited antigens: HL60 cells, Staphylococcus aureus, Coxiella burnetii, Legionella pneumophila, Bartonella henselae, Bartonella quitana, Bartonella vinsonii subsp. berkoffii and Bartonella elizabethae in example 3.

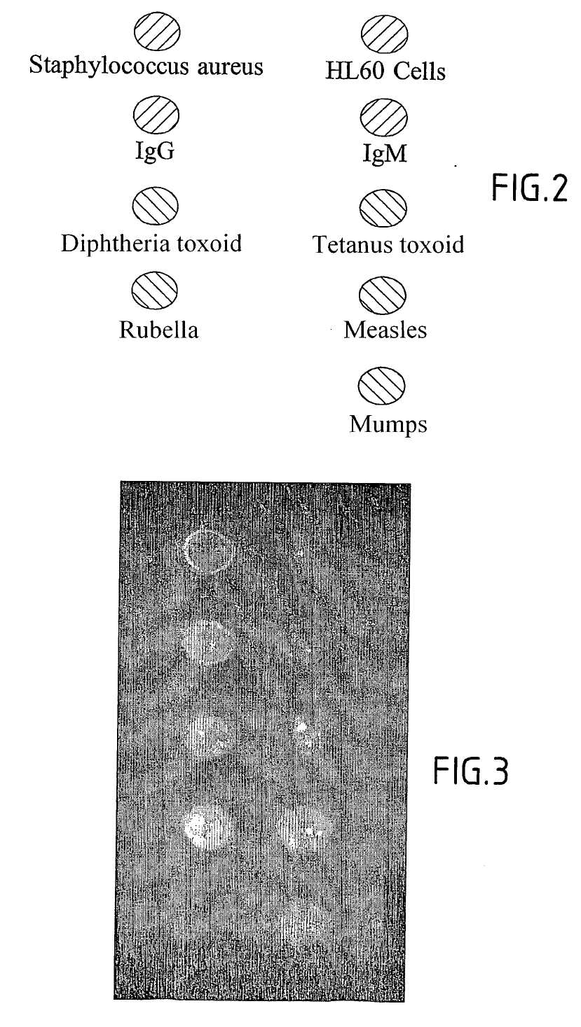

FIG. 2 is a schematic showing the layout of antigen deposits on a slide used in example 4.

FIG. 3 is a picture of the slide in example 4 and FIG. 5, after incubation with four sera, obtained at 350 nanometres (non-specific fluorescent labelling, with non-specific fluorescent marking of the proteins with AMCA and of DNA with HOESCHT 332-42 dye), according to example 4.

### EXAMPLE 1

### Depositing of Said Second and Fourth Antigens on a Solid Support

HL60 cells (ATCC N° CCL 240) are continuous line human fibroblast cells used to detect anti-nuclear antibodies. After culture and growing as per usual protocols, the concentration of HL60 cells was quantified using a cell counter (Microcytes® BPC/Yeast, BioDETECT AS, Oslo, Norway) and this concentration was brought to 10⁸ cells/mL in a sterile PBS buffer pH 7.4, to obtain a suspension of non-confluent cells able to be deposited by robot.

Staphylococcus aureus (ATCC deposit n° 29213) was cultured on agar gel with 5% sheep blood then harvested in sterile PBS buffer containing 0.1% sodium azide. The inoculum was measured using the same cell counter and calibrated at 10⁹ bacteria/mL which is the optimum concentration having regard to constraints of absence of sedimentation during deposit and the depositing of staphylococcal particles in sufficient quantity, then preserved by freezing at −80° C.

These HL60 cells and Staphylococcus aureus bacteria were deposited at a concentration of 10⁹ CFU/mL determined by FACS-scan (Microcytes®) on glass slides (Reference LLR2-45, CML, Nemours, France). The cells and bacteria were deposited on the solid support using a robot arrayer (Arrayer 427™ Affymetrix, MWG Biotech SA, Courtebeouf, France). The deposits were air dried for 30 minutes in the spotter chamber then fixed in a 100% methanol bath for 10 minutes, and dried again in open air. The efficiency of robot arrayer depositing on slides, after fixing in ethanol and after the baths required for the subsequent indirect immmunofluorescence reaction, was successfully verified by staining with the Hoescht 333-42 fluorescent dye which is excited at 350 nanometres and emits at 460 nanometres (Molecular Probes). FIG. 1 illustrates robot array depositing of 6 columns with 6 deposits each of Staphlyococcus aureus.

### EXAMPLE 2

### Depositing of Said First Antigen (IgG) and Said Third Antigen (IgM) on a Solid Support

Class G γ-specific human immunoglobulins(IgG), [Serotec, Cergy Saint-Christophe, France] diluted to a concentration of 5 mg/mL and class M γ-specific immunoglobulins (IgM) [Serotec] diluted to a concentration of 10 mg/mL to obtain fully homogeneous spots were robot deposited (Arrayer model 427, Affymetrix Inc. CA) on a solid support consisting of a glass slide (Reference LLR2-45, CML, Nemours, France).

The deposits were made by transfer of the antigen suspension from a microtitre plate containing 25 μL suspension, in a deposited volume of 1 mL, at 25° C. and 55% humidity in the chamber of the arrayer. These conditions were controlled by a thermo-hygrometer. The deposits of size 200 μm were air dried for 30 minutes at 37° C. in the arrayer chamber, then fixed in a bath of 100% ethanol for 10 minutes, and dried again in open air. The efficiency of arrayer depositing and of ethanol fixing after the baths required by the indirect immunofluorescence reaction was verified using the indirect immunofluorescence technique.

10 human sera containing rheumatoid factors and 10 human sera not containing rheumatoid factors (negative controls) for the immunodetection of rheumatoid factors [qualitative detection using the sensitized latex bead agglutination test (Rhumalatex Fumouze®, Laboratoires Fumouze, Levallois-Perret France)] were each deposited on an IgG deposit and each in three dilutions: 1:32, 1:64 and 1:128. After a first washing an indirect immunofluorescence reaction was conducted using goat immunoglobulin (Reference A-11013, Molecular Probes, Eugene, USA) as secondary anti-human IgM antibody, coupled to Alexa 488 which is a fluorescent molecule excited at 488 nanometres and emitting at 540 nanometres, and using the same immunoglobulin coupled to Axa 594 (A-21216 Molecular Probes, Eugene, USA) which is a fluorescent molecule excited at 594 nanometres and emitting at 640 nanometres in a second experiment. The reading of the reaction was made under fluorescence microscope and showed fluorescent detection in all sera containing rheumatoid factors in the form of a very shiny spot for each of the 3 tested dilutions at the Ig₁ deposits, and no fluorescence in the sera not containing these factors.

This example also illustrates that it is possible to conduct robot depositing of human IgG and IgM on a solid support under conditions compatible with the performing of indirect immunofluorescence reaction for the detection of rheumatoid factors. This example also illustrates that it is possible to perform the detection of rheumatoid factors using an indirect immunofluorescence technique.

Three IgM concentrations were deposited, from top to bottom: 10 mg/ml, 1 mg/ml and 0.2 mg/ml, six deposits were made per concentration. The concentration of 10 mg/ml gave the most homogeneous spots.

### EXAMPLE 3

### Serological Diagnosis of Infection with Coxiella burnetii Using the Method Described in the Present Invention

To conduct the different serological tests, 8 antigens were used: HL60 cells (as said second antigen), Staphylococcus aureus (as said fourth antigen), human IgGs (as said first antigen) and Coxiella burnetii Nine Mile, phase II (ATCC N° VR 616), Legionella pneumophila, Bartonella henselae, Bartonella quintana, Bartonella vinsonii subsp. berkoffii, and Bartonella elizabethae (as said microbial antigens).

After culture and production as per usual protocols, the concentration of the HL60 cells was quantified using a cell counter (Microcytes® BPC/Yeast, BioDETECT AS, Oslo, Norway) and this concentration was calibrated at 10⁷ cells/mL in a sterile PBS buffer pH 7.4 to obtain a suspension of non-confluent cells able to be deposited by robot arrayer. These cells were preserved in 100% ethanol at +4° C. before robot depositing.

Staphylococcus aureus was cultured on agar gel with 5% sheep blood, then harvested in sterile PBS buffer containing 0.1% sodium acid. The inoculum was measured using the same cell counter and calibrated at 10⁹ bacteria/mL then preserved by freezing at −80° C.

Class G human immunoglobulins (IgG) [Serotect, Cergy Saint-Christophe, France] were extemporaneously diluted at a concentration of 5 mg/ml.

Finally the bacterium Coxiella burnetii Nine Mile phase II was cultured on a cell support and purified following usual protocols and quantified with the Microcyte® counter to standardize its concentration at 10⁹ bacteria/mL which is the optimum concentration having regard to the constraints of no sedimentation during depositing and the deposit of bacterial particles in sufficient quantity. This antigen was preserved at −80° C. in PBS buffer, 0.1% sodium acid.

These 4 antigens were deposited on the solid support (Reference LLR42-45, CML, Nemours, France) using a robot arrayer (Arrayer 427™, Affymetrix, MWG Biotech SA, Courtaboeuf, France).

The solid support consisted of a glass slide previously cleaned with a 50% ethanol-50% acetone mixture, then dried in ambient air. The deposits were dried in air for 30 minutes in the chamber of the robot arrayer then fixed in a bath of 100% methanol for 10 minutes, and again dried in open air. The deposits were made by transfer of the suspension of antigen from a well of a microtitre plate containing 25 μL suspension, under a deposited volume of 1 mL, at 25° C. and 55% humidity in the chamber of the robot arrayer. These conditions were controlled with a thermo-hygrometer. The deposits of size 200 μm were dried in air for 30 minutes at 37° C. in the chamber of the robot arrayer then fixed in a bath of 100% methanol for 10 minutes and again dried in open air.

To perform the serological test, 5 human sera containing rheumatoid factors, 5 human sera containing anti-nuclear antibodies, 10 human sera containing IgG antibodies of anti-Coxiella burnetii phase II type at a titre of ≧1:200 as well as IgM antibodies of anti-Coxiella burnetii phase II type at a titre of ≧1:50, and 10 negative control human sera not containing any of these 3 types of antibodies were used and each deposited on an IgG deposit in three dilutions each: 1:32, 1:64 and 1:128.

After a first washing, indirect immunofluorescence reactions were conducted using:

a first secondary antibody consisting of goat anti-human IgM immunoglobulin, coupled to Alexa 488 (A-11013, Molecular Probes, Eugene, USA) which is a fluorescent molecule excited at 488 nanometres and emitting at 540 nanometres, and

a second secondary antibody consisting of goat anti-human IgG immunoglobulin(A-21216 Molecular Probes, Eugene USA) which is a fluorescent molecule excited at 594 nanometres and emitting at 640 nanometres in a second experiment.

These reactions were conducted at ambient temperature in a humid chamber, comprising 15 minutes incubation with the serum to be tested, followed by washing in sterile PBS buffer then incubation for 5 minutes with the conjugate antibodies before a second rinsing. Reading of the reactions was made under fluorescence microscope and gave rise to digitized recording of the spots. During this test:

the 30 sera “illuminated” the deposit of S. aureus i.e. reacted with said fourth antigen and said goat anti-human IgG coupled to Alexa 594.

only the sera containing rheumatoid factors “illuminated” the IgG deposit during reading of the Alexa 488 marker of said anti IgM immunoglobulin,

only the sera containing anti-nuclear antibodies “illuminated” the deposit of HL60 cells during reading with said second anti IgG detection substance coupled to Alexa 594, and

only those sera containing antibodies against C. burnetii “illuminated” the deposit of C. burnetii during reading with either of the two anti-IgM and anti-IgG detection substances,

all the reactions using conjugate anti-IgG secondary antibodies “illuminated” the IgG deposit.

The observed titres of anti-C. burnetii antibodies agreed with those found by the referenced method (Micro-immunofluorescence indirecte Dupont H T, Thirion X, Raoult D Q, fever serology: cutoff determination for microimmuno-fluorescence Clin. Diag. Lab Immunol. 1994;1:189-96).

FIG. 4 shows a slide stained with the Hoescht 333-42 fluorescent dye which is excited at 350 nanometres and emits at 460 nanometres (Molecular Probes) allowing visualization of all 8 deposited antigens: HL60 cells, Staphylococcus aureus, Coxiella burnetii, Legionella pneumophila, Bartonella henselae, Bartonella quintana, Bartonella vinsonii subsp. berkoffii, and Bartonella elizabethae. These different antigens were deposited under the same conditions as for Coxiella described above.

### EXAMPLE 4

### Determination of a Person's Vaccine Status

A glass slide was developed for determining a person's vaccine status, comprising a total of 8 deposits of antigens containing 3 control deposits and 5 vaccine antigen deposits on one same slide, divided into 2 columns. The conditions of deposit and use of the 3 control deposits containing Staphylococcus aureus, IgG and HL60 cells were set forth under examples 1 and 2 above.

The deposits of vaccine antigens were made in the form of a viral suspension of whole inactivated living viruses deposited at a concentration of 10⁹ or 10¹⁰ particles/ml in a mixture with goat IgG at a concentration of 25 to 75% (volume/volume) and in accordance with the following conditions:

(1) Rubella antigen: the strain used was hpv-77 (Microbix Biosystems Inc., Toronto, Canada) supplied in the form of an inactivated viral suspension at a protein concentration of 0.51 mg/ml. This antigen was concentrated 10 times by centrifuging before being deposited in a volume of 45 pL antigen and 5 μL goat IgG immunoglobulin at a titre of 10 mg/ml (reference I5256, Sigma, Saint-Quentin Fallavier, France), stained with 2 μL amino methyl coumarin acetyl (AMCA) (Molecular Probes, Interchim, Montlucon France).

AMCA binds non specifically to the proteins of this antigen and can therefore be used to verify depositing of the antigen on the solid support by physical adsorption. It is excited at the same wavelength of 350 nm as the Hoescht 333-42 dye.

(2) Measles antigen: the strain used was Edmonston (Microbix Biosystems Inc.) supplied in the form of a suspension of inactivated viral particles at a protein concentration of 1.95 mg/ml which was concentrated 5 times by centrifuging before being deposited in a volume of 45 μL antigen and 5 μL goat IgG immunoglobulin stained with AMCA.

(3) Mumps antigen: the strain used was Enders (Microbix Biosystems Inc.) with a protein concentration of 1.8 μg/ml and deposited in a volume of 45 μL antigen and 5 μL goat IgG immunoglobulin labelled with AMCA.

(4) Diphtheria toxoid (reference FA 150934, Aventis Pasteur Vaccins) with a protein concentration of 18.9 mg/ml and concentrated 10 times before being deposited in a volume of 45 μL antigen and 5 μL goat IgG immunoglobulin labelled with AMCA.

(5) Tetanus toxoid (reference J001, AVentis PAsteur vaccines) at a concentration of 80 IU/mL and deposited in pure form in a volume of 45 μL antigen and 5 μL goat IgG immunoglobulin labelled with AMCA.

The deposits of antigens were made in a volume of 1 nl using an Affymetrix spotter of a 427 Arrayer. After drying, the slides so prepared were used as support for an indirect immunofluorescence reaction for the detection of IgGs specific to the vaccine antigens in the sera of individuals, as per the following protocol:

during a first step, 5 μM of serum taken by vein puncture or capillary puncture are contacted and incubated with the slide at the different vaccine antigens and control antigens, using an automated incubator,

during a second step the automated incubator rinses the slide to remove the tested serum and performs incubation with the anti-human IgG detection antibody conjugated with fluorescein which is excited at a wavelength of 488 nm (reference Star 106 F, Serotec, France).

during a third step, the automated incubator rinses the slide to remove the conjugate detection antibody and dries the slide,

during a fourth step, the slide thus treated is removed from the automated incubator and placed in the reading chamber of an automatic fluorescence reader. This reader successively makes two readings, one at 350 nm which is the emission wavelength of the AMCA dye then a second reading at 488 nm which is the emission wavelength of the fluorescein of the detection antibody,

during a fifth step, this data is transmitted to software to be converted into fluorescence intensity at 350 nm and at 488 n for each of the deposits,

during a sixth step, the software analyses the fluorescence intensities and successively verifies:
the presence of fluorescence for the deposit of Staphylococcus aureus to check the presence of the serum to be tested;
the presence of fluorescence for the IgG deposit to check the quality of the reaction involving the conjugate detection antibody,
the absence of fluorescence for the deposit of HL60 cells to check the presence or absence of anti-nuclear antibodies in the serum to be tested,

during a seventh step, the software calculates the fluorescence ratio 488/350 for each of the vaccine antigen deposits and for each vaccine antigen deposit compares this value with a previously determined ratio curve for each vaccine antigen, determined using positive control sera containing specific antibodies at a known concentration using a reference method, and negative sera not containing specific antibodies detectable by a reference method.

Fluorescence at 350 nm results from excitation of the non-specific markers which bind non-specifically either to the DNA or to the proteins of the vaccine antigens. The quantity of fluorescence emitted by the antigen deposits at 350 nm is proportional to the quantity of antigens effectively present in the deposit, which means that this fluorescence at 350 nm is a measurement which is dependent upon the quantity of antigens present in the deposit. The quantity of fluorescence at 350 nm is used by the software:

firstly to identify the topographical position of the antigen deposits on the slide, and

secondly to quantify their exact surface area and hence the contours in order not to incorporate fluorescence artefacts which may lie outside these spots, and

finally to weight fluorescence at 488 nm. This second wavelength of 488 nm results from excitation of the marker of the detection G immunoglublin. The quantity of fluorescence at 488 nm relates to the quantity of specific immunoglobulin G present in the spot of the patient's serum to be tested. Fluorescence at 488 nm (and hence the fixing of G immunoglobulins) depends upon the quantity of antigens which have been deposited. If very few antigens are deposited, the quantity of fixed specific immunoglobulin will be low and hence the fluorescence at 488 nm will be low. This is why the quantity of fluorescence at 488 nm is weighted for each of the spots of vaccine antigens by the quantity of fluorescence at 350 nm and by the fluorescence ratio expressing the quantity of specific IgGs present in the tested serum,

during a final step, the software interprets the results by comparing each of the 488/350 fluorescence ratios measured for the tested sera with those obtained from a collection of positive and negative reference sera used to determine the reference curves, all this data being used to indicate the list of vaccine antigens against which the tested serum contains specific antibodies, and hence giving the vaccine status of the person.

Therefore in this example four sera were tested taken from four different patients to identify the presence of class IgG antibodies against the vaccine antigens of measles, rubella, mumps, diphtheria and tetanus. The threshold value of the fluorescence ratio was determined for each of these 5 vaccine antigens.

Tables 1A, 1B, 1C and 1D show the fluorescence ratios for each of the 4 tested sera respectively.

The reading of tables 1A to 1C indicates that:

serum n°1 is:
positive for the presence of diphtheria anti-toxoid antibodies (fluorescence ratio >1, threshold value),
positive for the presence of tetanus anti-toxoid antibodies (fluorescence ratio >0.05, threshold value),
negative for the presence of anti-rubella antibodies (fluorescence ratio <0.1, threshold value),
positive for the presence of anti-measles antibodies (fluorescence ratio >0.1, threshold value)
positive for the present of anti-mumps antibodies (fluorescence ratio >0.11, threshold value);

serum n° 2 is:
positive for the present of diphtheria anti-toxoid antibodies (fluorescence ratio >1, threshold value),
positive for the presence of tetanus anti-toxoid antibodies (fluorescence ratio >0.05, threshold value)
positive for the present of anti-rubella antibodies (fluorescence ratio >0.1, threshold value),
positive for the presence of anti-measles antibodies (fluorescence ratio >0.1, threshold value), and
positive for the presence of anti-mumps antibodies (fluorescence ratio >0.11, threshold value);

serum n° 3 is:
negative for the presence of diphtheria anti-toxoid antibodies (fluorescence ratio <1, threshold value),
negative for the presence of tetanus anti-toxoid antibodies (fluorescence ratio <0.05, threshold value),
negative for the presence of anti-rubella antibodies (fluorescence ratio <0.1, threshold value),
positive for the presence of anti-measles antibodies fluorescence ratio >0.1, threshold value),
negative for the presence of anti-mumps antibodies (fluorescence ratio <0.11, threshold value);

serum n°4 is:
negative for the presence of diphtheria anti-toxoid antibodies (fluorescence ratio <1, threshold value),
negative for the presence of tetanus anti-toxoid antibodies (fluorescence ratio <0.05, threshold value),
positive for the presence of anti-rubella antibodies (fluorescence ratio >0.1, threshold value),
negative for the presence of anti-measles antibodies (fluorescence ratio <0.1, threshold value),
negative for the presence of anti-mumps antibodies (fluorescence ratio of 0.11, threshold value).

FIG. 2 is a schematic giving the layout of antigen deposits on the vaccine slide.

FIG. 3 is the image of this slide after incubation with the 4 above-cited sera, obtained at 350 nm (non-specific fluorescent staining after non-specific fluorescent labelling of proteins with AMCA and of DNA with Hoescht 332-42 dye). This image is used to control the presence of each of the deposits of control antigen and vaccine antigen.

FIGS. 2 and 3 show an IgM spot (deposit area) which was not used in the tests performed.

TABLE 1A
Serum n° 1

| | | | Surface | | Ratio | Fluorescence |
|---|---|---|---|---|---|---|
| Number | Abscissa | Ordinate | area | Fluorescence 350 | F350/F488 | 488 |
| SA | 240 | 127 | 1228 | 44238 | 1.274 | 56359 |
| IgG | 238 | 195 | 1198 | 54695 | 0.902 | 49335 |
| Diphtheria | 238 | 263 | 1203 | 44069 | 1.691 | 74521 |
| Rubella | 235 | 332 | 1117 | 56298 | 0.075 | 4222 |
| HL | 309 | 123 | 452 | 9652 | 0.458 | 4421 |
| IgM | 306 | 197 | 918 | 18369 | 0.183 | 3362 |
| Tetanus | 304 | 266 | 842 | 26512 | 0.078 | 2068 |
| Measles | 303 | 334 | 1104 | 37075 | 0.13 | 4820 |
| Mumps | 302 | 401 | 1038 | 29978 | 0.118 | 3537 |

TABLE 1B
Serum n°2

| | | | Surface | | Ratio | Fluorescence |
|---|---|---|---|---|---|---|
| Number | Abscissa | Ordinate | area | Fluorescence 350 | F350/F488 | 488 |
| SA | 249 | 109 | 1199 | 41653 | 1.839 | 76600 |
| IgG | 250 | 177 | 1255 | 49420 | 1.263 | 62417 |
| Diphtheria | 251 | 247 | 1176 | 39874 | 2.350 | 93704 |
| Rubella | 251 | 313 | 1173 | 50395 | 0.108 | 5443 |
| HL | 316 | 106 | 537 | 10853 | 0.627 | 6805 |
| IgM | 318 | 176 | 895 | 15917 | 0.249 | 3963 |
| Tetanus | 321 | 244 | 834 | 21013 | 0.357 | 7502 |
| Measles | 319 | 314 | 1164 | 39486 | 0.118 | 4659 |
| Mumps | 320 | 382 | 1079 | 31342 | 0.120 | 3761 |

TABLE 1C
Serum n°3

| | | | Surface | | Ratio | Fluorescence |
|---|---|---|---|---|---|---|
| Number | Abscissa | Ordinate | area | Fluorescence 350 | F350/F488 | 488 |
| SA | 210 | 85 | 1175 | 34462 | 1.981 | 68269 |
| IgG | 210 | 153 | 1293 | 47678 | 1.561 | 74425 |
| Diphtheria | 210 | 220 | 1255 | 54523 | 0.342 | 18647 |
| Rubella | 209 | 288 | 1172 | 49101 | 0.073 | 3584 |
| HL | 278 | 78 | 474 | 9180 | 0.740 | 6793 |
| IgM | 278 | 151 | 943 | 15225 | 0.335 | 5100 |
| Tetanus | 278 | 220 | 848 | 24553 | 0.046 | 1129 |
| Measles | 278 | 288 | 1178 | 34879 | 0.153 | 5336 |
| Mumps | 278 | 357 | 1123 | 26949 | 0.095 | 2560 |

TABLE 1D
Serum n°4

| | | | Surface | | Ratio | Fluorescence |
|---|---|---|---|---|---|---|
| Number | Abscissa | Ordinate | area | Fluorescence 350 | F350/F488 | 488 |
| SA | 240 | 110 | 1202 | 40889 | 1.093 | 44692 |
| IgG | 240 | 177 | 1269 | 53557 | 1.123 | 60145 |
| Diptheria | 242 | 246 | 1253 | 56284 | 0.316 | 17786 |
| Rubella | 241 | 314 | 1106 | 43970 | 0.107 | 4705 |
| HL | 308 | 106 | 533 | 9932 | 0.797 | 7916 |
| IgM | 308 | 178 | 926 | 17540 | 0.309 | 5420 |
| Tetanos | 309 | 244 | 838 | 29892 | 0.049 | 1465 |
| Measles | 309 | 314 | 1174 | 41689 | 0.093 | 3877 |
| Mumps | 310 | 382 | 1044 | 29666 | 0.110 | 3263 |

1. In vitro serological method for diagnosing microbial agents by immunodetection, wherein the presence is detected and, preferably, the quantity of patient immunoglobulins is assayed of both classes M and G, or only class G, specific to a microbial antigen characteristic of said microbial agent, in a patient's serum sample to be tested, by detection and quantification of an immunological reaction complex between said microbial antigen to be detected and a said specific class M immunoglobulin for IgM assay and/or respectively a said specific class G immunoglobulin for IgG assay, using a first detection substance and/or respectively a second detection substance comprising an antibody only reacting with a said immunoglobulin of the patient species of class M and/or respectively G, characterized in that:
1/The steps are performed in which:
said serum sample to be tested is contacted with said first and second detection substances, or only said second detection substance, and with at least one solid support on which the following antigens have been attached:
a first control antigen consisting of a non-specific class G immunoglobulin of the patient species, and
a second control antigen containing DNA/histone complexes,
optionally, a third control antigen consisting of a non-specific class M immunoglobulin of the patient species, the presence of said third control antigen being necessary in the event of IgM assay, and
at least one said microbial antigen, and
a series of controls is conducted comprising:
a—controlling the reactivity of said second detection substance by verifying whether said first control antigen reacts with said second detection substance, and optionally control of the presence of rheumatoid factors in said serum sample by verifying whether the first control antigen reacts with said serum sample and said first detection substance, in the event of IgM assay,
b—controlling the presence of anti-nuclear antibodies in said serum sample to be tested by verifying whether said second control antigen reacts with said serum sample and second detection substance,
c—controlling the reactivity of said first detection substance by verifying whether said third control antigen reacts with said first detection substance, in the event of IgM assay, and
d—controlling the presence of a human serum in the sample to be tested, and
2/A reaction result between said microbial antigen, said serum sample and a said detection substance is only taken into account if the control of the presence of a human serum is positive and if the following accumulative conditions are met:
a—said first control antigen reacts with said second detection substance,
b—said second control antigen does not react, and
c—when applicable, said third control antigen reacts with said first detection substance, in the event of IgM assay.

2. Method as in claim 1, characterized in that a single solid support is used, contacted optionally simultaneously with said first and second detection substances containing a first and respectively a second labelling element, the second labelling element emitting a different signal to the first labelling element, said first and second detection substances containing a first and respectively a second antibody only reacting with a said immunoglobulin of the patient species of class M and respectively class G.

3. Method as in claim 1, characterized in that it is controlled that said tested sample does contain a serum of the patient species by detecting whether immunoglobulins of the patient species react with a fourth control antigen containing protein A of a Staphylococcus aureus bacterium, preferably said fourth antigen being a whole Staphylococcus bacterium, by contacting said sample with a solid support on which a said fourth control antigen is attached in the presence of said second detection substance which is an anti-immunoglobulin antibody of the patient species not reacting with said fourth control antigen, the control of the presence of a serum being positive if said fourth antigen reacts with said serum sample and said second detection substance.

4. Method as in claim 1, characterized in that said second detection substance is an animal immunoglobulin, preferably a goat or chicken immunoglobulin.

5. Method as in claim 1, characterized in that, optionally, the two said first and second detection substances are goat or chicken immunoglobulins, respectively anti-IgM and anti-IgG.

6. Method as in claim 1, characterized in that said second control antigen consists of non-confluent human fibroblast cells in suspension.

7. Method as in claim 1, characterized in that said control antigens and microbial antigens are attached to the solid support by physical adsorption.

8. Method as in claim 1, characterized in that said microbial antigen is a corpuscular antigen consisting of a whole inactivated microbe or microbe fraction.

9. Method as in claim 1, characterized in that said microbial agent is chosen from among the micro-organisms comprising a bacterium, a virus, a parasite or a fungus.

10. Method as in claim 9, characterized in that said microbial antigen is an intracellular bacterium or a virus.

11. Method as in claim 9, characterized in that said microbial antigen is chosen from among the bacteria of genus Rickettsia, Coxiella, Bartonella, Tropheryma, Ehrlichia, Chlamydia, Mycoplasma, Treponema, Borrelia and Leptospira.

12. Method as in claim 11, characterized in that said microbial antigen is a bacterium responsible for endocarditis.

13. Method as in claim 10, characterized in that said microbial antigen is a viral antigen chosen from among the viruses H.I.V, C.M.V., Epstein-Barr, Measles, Rubella, Hepatitis A and B.

14. Method as in claim 1, characterized in that:
the detection, and the assay is performed of the quantity of patient immunoglobulins of both classes M and G specific to a microbial antigen,
at least one said microbial antigen and said first, second and third and fourth control antigens are attached to one same solid support, and
for the detection of the different said microbial antigens, the same said first and second detection substances are used with different labelling elements, said first and second detection substances being animal immunoglobulins not reacting with said fourth antigen.

15. Method as in claim 1, characterized in that as solid support a glass or plastic slide is used, or a titre tube or well of a plastic microtitre plate.

16. Method as in claim 1, characterized in that in the sample to be tested the detection and, optionally the assay of said immunoglobulin of the patient species specific to said microbial antigen is performed, and the immunological reactions between said control antigens and said detection substances are read by automated reading using equipment for reading a fluorescent signal of a fluorescent substance corresponding to the labelling elements of said detection substances.

17. Method as in claim 1, characterized in that said microbial antigen is a vaccine antigen and said immunoglobulin specific to said vaccine agent to be detected is a class G immunoglobulin.

18. Method as in claim 17, characterized in that the vaccine status of a person is determined by detection, preferably quantification of IgG serum antibodies specific to the vaccine antigens of a plurality of pathogenic agents of bacterial, viral, fungal or parasitic type, by detecting, and preferably quantifying, an immunological reaction complex between each said vaccine antigen and respectively each said antibody specific to said vaccine antigen, which may be present in a human serum sample to be tested, comprising:
1. Contacting one single, same said serum sample to be tested with:
one same solid support on which a plurality of said vaccine antigens is attached corresponding to a plurality of pathogenic agents, and said first, second and preferably fourth control antigens,
in the presence of at least one said second detection substance reacting with at least one said specific antibody and not reacting with any of said vaccine antigens, and
2. Performing at least one said control of the reactivity of said second detection substance using a said first control antigen, and one said control of the presence of anti-nuclear antibodies using at least one said second control antigen, and controlling the presence of human serum in said sample to be tested.

19. Method as in claim 18, characterized in that said specific antibodies of IgG immunoglobulin type are detected and a said second detection substance is used which is an antiIgG immunoglobulin, consisting of a goat or chicken immunoglobulin.

20. Method as in claim 18, characterized in that said vaccine antigens are antigens of pathogenic agents chosen from among the viruses of mumps, rubella, measles, chicken pox, poliomyelitis, yellow fever, tick-borne encephalitis, hepatitis A, hepatitis B and the bacteria of Bordetella pertussis, tetanus and diphtheria.

21. Method as in claim 18, characterized in that it is determined whether the concentration of said specific antibodies reaches a threshold on and after which said specific antibody has a protective action protecting against the disease determined by the pathogen.

22. Method as in claim 18, characterized in that a determined volume of whole blood is collected using a capillary tube in a flask containing a determined volume of buffer allowing elution of the serum, the serum then preferably being diluted to a determined concentration, preferably 1:100 to 1:20.

23. Method as in claim 18, characterized in that, for each detection and optionally quantification of a said vaccine antigen, the following measurements are made:
1—a first measurement of a first value representing the quantity of a first labelling element, said first value being the intensity value of a signal emitted by said first fluorescent labelling element, said first labelling element binding itself non specifically to any protein in the depositing area of said vaccine antigen, and
2—a second measurement of a second value representing the quantity of a second labelling element emitting a different signal to said first labelling element, said second value being the intensity value of the signal emitted by this second fluorescent labelling element at a different excitation wavelength to that of said first fluorescent labelling element, said second labelling element being the labelling element of said second detection substance for said vaccine antigen in the depositing area of said antigen, and
3—the ratio between said first and second values is calculated, and
4—the value of said ratio is compared with the value of a reference ratio obtained with a collection of positive and negative reference sera, thereby making it possible to determine, by comparison, whether or not it is necessary to vaccinate the person against said vaccine antigen in relation to the ratio between said first and second values.

24. Diagnosis kit which can be used for implementing a method as in claim 1, characterized in that it contains:
at least one said solid support on which at least one said microbial antigen and said control antigen(s) are attached, said control antigens containing at least said first and second control antigens and optionally said third and fourth control antigens, and
said detection substance(s) and reagents used to develop said labelling element(s).

25. Kit as in claim 24, characterized in that it contains:
one same said solid support on which at least one said corpuscular antigen and said control antigens are attached by physical adsorption, and
at least one of a same said first or second detection substance to detect the different microbial antigens.

26. Kit as in claim 24, characterized in that it comprises a flask containing a determined volume of elution buffer to collect a determined volume of serum sample to be tested.

27. Method for preparing a kit as claimed in claim 23 comprising a solid support on which at least one antigen is attached chosen from among a said microbial agent, preferably corpuscular, a said first, a said second, optionally a said third a said fourth control antigen enabling detection by automated reading using a said first and optionally said second detection substance, characterized in that said microbial antigens, preferably corpuscular, and control antigens are deposited by robot arrayer which preferably comprises a syringe, said corpuscular antigens preferably being associated with a dye, preferably a fluorescent dye in the form of a suspension at a concentration enabling their visualization with said dye after being deposited, thereby making it possible to verify the attachment of said antigens to said solid support.

28. Method as in claim 27, characterized in that a robot arrayer is used to deposit a said microbial antigen and optionally a said second control antigen and optionally a said fourth control antigen in the form of a suspension of non-confluent cell corpuscles, whole viruses or whole bacteria, or fractions of cells or bacteria.

29. Method as in claim 28, characterized in that the control antigens in the form of a cell suspension are calibrated at a concentration of 10⁷ to 10⁹ cells/ml, said control antigens or microbial antigens in the form of suspensions of bacteria or bacteria fractions are calibrated at a concentration of 10⁷ to 10⁹ particles/ml, and the suspensions of whole viruses at a concentration of 10⁹ to 10¹⁰ particles/ml.

30. Method as in claim 28, characterized in that said control antigens and corpuscular microbial antigens are deposited in a mixture with a protein binder to stabilize attachment to said solid support.

31. Method as in claim 30, characterized in that said protein binder is chosen from among egg yolk, gelatine, bovine serum albumin or a non-human polyclonal IgG, preferably goat.

32. Method as in claim 31, characterized in that said corpuscular microbial antigen is deposited on said solid support consisting of a glass slide, in a mixture with an immunoglobulin of goat polyclonal IgG type.

33. Method as in claim 27, characterized in that prior washing of said solid support is performed with a solution of an ethanol/acetone mixture, preferably 50-50, then the said antigens are deposited and their attachment stabilized by physical adsorption on said solid support by treatment with alcohol, preferably methanol or ethanol, which alcohol is subsequently removed, further preferably the attachment of said antigens is verified by staining, preferably with fluorescent labelling non-specific to the proteins or DNA.

34. Method as in claim 27, characterized in that the attachment by physical adsorption of said control antigens and microbial antigens is completed by cross-linking treatment, preferably a chemical treatment using a bi-functional agent for covalent coupling.
